(19) United States
(12) Patent Application Publication
(10) Pub. No.: US 2014/0343326 A1
(43) Pub. Date: Nov. 20, 2014

(54) FAST CATALYZED HYPOHALOUS OXIDATION OF ALCOHOL GROUPS

(71) Applicant: Corning Incorporated, Corning, NY (US)

(72) Inventors: Olivier Gaurat, Avon (FR); Clemens Rudolf Horn, Guibeville (FR); Patrick Jean, Le Chatelet En Brie (FR)

(21) Appl. No.: 14/372,289

(22) PCT Filed: Jan. 17, 2013
(86) PCT No.: PCT/US2013/021808

§ 371 Date: Jul. 15, 2014

(30) Foreign Application Priority Data
Jan. 17, 2012 (EP) 12305062.7

(51) Int. Cl. C07C 45/30

(52) U.S. Cl. CPC C07C 45/30; C07B 41/06
USPC 568/426; 568/485; 568/420

(57) ABSTRACT

An improved process for oxidation of at least one alcohol group of at least one chemical compound to the corresponding carbonyl group. Said process is carried out in the presence of a buffered oxidative hypohalous solution and of a nitroxide oxidation catalyst. It is characteristically carried out within a micro-reactor; the buffered oxidative hypohalous solution being extemporaneously prepared at a buffered pH comprised between 7 and less than 8.5 (7≦pH<8.5).

This application claims the benefit of priority under 35 U.S.C. §119 of European Patent Application Serial No. 12305062.7 filed on Jan. 17, 2012 the content of which is relied upon and incorporated herein by reference in its entirety.

### FIELD

The present disclosure concerns a process for oxidation of alcohols to aldehydes and ketones. It more particularly concerns an improved process for oxidation of at least one alcohol group of at least one chemical compound to the corresponding carbonyl group, in the presence of a buffered oxidative hypohalous solution and a nitroxide oxidation catalyst, such as TEMPO ((2,2,6,6-tetramethylpiperidin-1-yl)oxyl).

### BACKGROUND

The TEMPO oxidation of alcohols to aldehydes and ketones was discovered in 1987 (P. L. Anelli et al. J. Org. Chem. 1987, 52, 2559-2562) (Annelli '87”). According to Anelli '87, the reaction is performed batchwise, at a lab scale, with freshly prepared bleach as oxidant (the problem of the degradation of bleach solutions [more and more critical as their pH decreases below 9.5] being well-known by one of skill in the art). It is more particularly performed at 0° C., at a pH of 8.6, with 1 mol % of TEMPO and 10 mol % of co-catalyst such as KBr.

Since 1987 TEMPO oxidation with a hypohalous solution has become increasingly popular, replacing other oxidations like, for example, PCC (Pyridine ChloroChromate) oxidation. As of 2011, nearly 20,000 reactions using TEMPO-type oxidations (carried out with hypochlorous solutions) have been published. The reaction conditions have not changed significantly since 1987, and are referred to as standard (see, e.g., R. Sheldon in Acc. Chem. Res. 2002, 35, 774; WO2010/023678). Other catalysts have been developed, notably polymer-bonded derivatives like PIPO (Polymer Immobilized TEMPO) (see, e.g., U.S. Pat. No. 6,995,291). The use of immobilized TEMPO in batch reactors and in micro-channels, respectively, is mentioned in U.S. Pat. No. 6,995,291 and in EP1534421. The U.S. Pat. No. 7,700,196 describes the use of TEMPO in range of 0.01 to 100 mol %, but preferably 0.1-2 mol %, to generate a carbonyl group in an organosilicon compound. TEMPO oxidation is considered as “part of the central tool box in the industrial organic chemistry” (R. Ciriminna et al. Org. Proc. Res. Dev. 2010, 14, 245).

The proposed and commonly accepted mechanism for the TEMPO reaction suggests that the use of the co-catalyst (such as KBr) accelerates the reaction. See the reaction diagram of FIG. 1A. As commonly understood, the reaction can work without co-catalyst but might take much longer, even when the temperature is increased such as to room temperature.

When carrying out the TEMPO reaction (a highly exothermic reaction) batchwise at larger scales, the bleach is slowly added and the stability of said bleach is a more critical problem. Accordingly, Anelli et al., Org. Synth. 1990, 69, 212-219 discloses carrying out said reaction at a pH of 9.5 (with the co-catalyst KBr).

Carrying out the TEMPO reaction in a micro-reactor or tube reactor is also known, applying the above mentioned conditions, or with even more catalyst (see, e.g., A. Bodgan et al. Beilstein J. Org. Chem. 2009, 5, 17; W. Ferstl et al. Chem. Eng. J. 2008, 135S, S292; E. Fritz-Langhals Org. Proc. Res. Dev. 2005, 9, 577). Fritz-Lanhghals in particular shows the industrial feasibility of this reaction in a tube reactor, with 25 sec residence time at a pH of 9.5 using 2 mol % of catalyst and 2 mol % of co-catalyst. The TEMPO-catalysed oxidation of alcohols by hypochlorite has also been carried out in a spinning tube-in-tube reactor (see P. D. Hampton et al Org. Proc. Res. Dev. 2008, 12, 946-949). The conditions of the reaction are very similar to the ones disclosed in Anelli '87. The bleach solution (commercial solution saturated in sodium bicarbonate) is freshly prepared, and a co-catalyst is used (Bu₄NBr, which acts both as co-catalyst (Br⁻) and as phase-transfer agent (Bu₄N⁺)). In view of the given results, the presence of Bu₄NBr appears to be a key point. The reaction is carried out at a lab scale and requires energy to spin the rotor of the reactor. No teaching in reference to the problem of the management of the feedstock of the oxidant solution (which is not stable at the indicated pH) is disclosed.

From a process point of view, alternative methods for continuous flow oxidation of alcohols and aldehydes (alternatives to the TEMPO oxidation) have been proposed:
a. a method disclosed in B. Leduc et al. Org. Proc. Res. Dev. 2012, 16 (5), 1082-1089, utilizing bleach and catalytic tetrabutylammonium bromide. The reaction conditions are mild and generally provide complete conversion in 5-30 min;
b. a method disclosed in U.S. Pat. No. 7,795,475 based on the elimination of H₂ in the presence of a copper catalyst in gas/liquid flow.

### SUMMARY

In such a context, the inventors propose an improved way of carrying out a TEMPO-type oxidation.

The improved TEMPO-type oxidation process of the present disclosure is hereafter described in detail.

A process for oxidation of at least one alcohol group (=at least one hydroxyl group) of at least one chemical compound to the corresponding carbonyl group is more generally concerned.

Said process can be carried out for the oxidation of a single chemical compound (with alcohol group(s) in its formula) or for the oxidation of (a mixture of) at least two chemical compounds (with alcohol group(s) in their formulae). It is generally carried out for the oxidation of a single chemical compound.

It has here to be indicated that the concerned chemical compound(s) with at least one alcohol group to oxidize according to the process of the present disclosure is(are) quite able to include, in addition to said at least one alcohol group to oxidize, at least another alcohol group, which is protected from oxidation and consequently not concerned by the oxidation process of the present disclosure.

In its (their) chemical formula (e), the concerned compound(s) has (have) at least one alcohol group (=at least one hydroxyl) group. It (they) actually has(have) at least one primary alcohol group (a single one or at least two), or at least one secondary alcohol group (a single one or at least two), or at least one primary alcohol group and at least one secondary alcohol group (in that later case, the concerned chemical compound(s) has(have) both primary alcohol group(s) and secondary alcohol group(s) in its(their) chemical formula (e)). It is here indicated that, in case of the presence of at least two alcohol groups to oxidize according to the present disclosure, it can sometimes be possible to carry out a selective oxidation, thanks to a setting of the residence time in the concerned reactor. So, for example, if primary and secondary alcohol groups are present, the process of the present disclosure can be carried out to oxidize both the primary and secondary alcohol groups or to oxidize only the primary alcohol group(s). This will addressed further later in the present text.

One skilled in the art knows that a primary alcohol group is oxidized to an aldehyde group while a secondary alcohol group is oxidized to a ketone group.

So the process of the present disclosure is suitable for the oxidation of a primary alcohol to an aldehyde and of a secondary alcohol to a ketone. It is more generally suitable to oxidize primary alcohol group(s) or secondary alcohol group(s) or both primary and secondary alcohol group(s) of a chemical compound or of a mixture of chemical compounds. The oxidation in question can be a selective one.

The oxidation process of the present disclosure is conventionally carried out in the presence of a buffered oxidative hypohalous solution (such as a bleach solution) and of a nitroxide oxidation catalyst (such as TEMPO). It conventionally takes place in a biphasic system. The used oxidant and catalyst are those of the prior art.

Characteristically, the process for oxidation (of at least one alcohol group of at least one chemical compound to the corresponding carbonyl group) of the present disclosure is carried out:
a. within a micro-reactor, and
b. with the buffered oxidative hypohalous solution extemporaneously prepared at a buffered pH comprised between 7 and less than 8.5 (7≦pH<8.5).

The inventors have shown that it is possible, using an extemporaneously prepared buffered oxidative solution and carrying out the reaction in a micro-reactor, to manage the oxidation reaction in pH conditions under which said buffered oxidative solution is known as being not stable and that then very interesting results are obtained: maximum yields are reached in a very short time (generally ≦3 min, indeed ≦1 min 30 s), i.e. a high rate of the oxidation reaction, and that without the use of any co-catalyst and at room temperature, indeed with the use of low quantity of catalyst (see further discussion below). In other words, the instability zone of the oxidative solution is a very favorable zone to carry out the reaction and it is possible to have the degradation rate of the oxidative solution slower than the reaction rate. Such a result was hard to foresee.

Characteristically, the process of the present disclosure is carried out inside a micro-reactor. Here is a key of said process. It is so carried out in a continuous way inside a micro-reactor. Micro-reactors are reactors well-known from one skilled in the art (see, e.g., N. Kockmann et al. Micro Process Engineering: Fundamentals, Devices, Fabrication, and Applications, Volume 5, 2006). They can be made from different materials such as stainless steels, plastics, glasses, glass-ceramics and ceramics. Micro-reactors can be obtained from different processes, more particularly from those described by the applicant in patent applications US2004/0206391 and WO2008/106160, for example. Micro-reactors are more and more used today. Their reactant passages, showing a circular or non-circular section, generally have a hydraulic diameter within the following range: 0.2-15 mm. The process of the present disclosure is carried out within such a micro-reactor, and it is advantageously carried out within a micro-reactor with reactant passage(s) showing a circular or non-circular section having a hydraulic diameter within the following range: 0.2-4 mm. The process of the present disclosure is obviously carried out in a micro-reactor, the constitutive material of which (or at least the material aimed to be in contact with the reactive medium of which) is compatible with the reactive medium. It is advantageously carried out in a micro-reactor made of glass, glass-ceramic or ceramic.

Characteristically, the process of the present disclosure is carried out using a buffered oxidative hypohalous solution extemporaneously prepared at a buffered pH comprised between 7 and less than 8.5 (7≦pH<8.5), as the oxidant.

The pH value of the buffered oxidative solution is another key of the process of the present disclosure. It is comprised between 7 and less than 8.5.

Said pH value is advantageously comprised between 7.5 and 8.4 (7.5≦pH≦8.4).

It is very advantageously comprised between 7.8 and 8.2 (7.8≦pH≦8.2). According to a particularly preferred embodiment, it is equal to 8. These very advantageous and particularly preferred ranges of the pH value are more particularly indicated in reference to the oxidation of primary alcohol group(s).

For the oxidation of secondary alcohol group(s), the pH value of the buffered oxidative hypohalous solution is generally advantageously comprised between 8 and 8.4 (8≦pH≦8.4).

The pH of the oxidative buffered hypohalous solution can have been adjusted to a desired value within the given range: from 7 to less than 8.5, thanks to any method well known to one skilled in the art.

The oxidative hypohalous solution used, which is buffered at a pH within the above indicated pH range, is extemporaneously prepared. Here is the last key of the process of the present disclosure. The extemporaneously preparation of the buffered oxidative hypohalous solution is required in view of the instability of said solution at said pH. The life time of the hypohalous solution in the pH range of 7 to less than 8.5 is actually limited. Thus, the prepared oxidative hypohalous solution has to be rapidly used (has to be rapidly reacted with the compound(s) having the alcohol group(s) in the presence of the catalyst). It is highly advised to use it in less than 10 minutes after its preparation, advantageously in less than 5 minutes and very advantageously in less than 3 minutes. All the used oxidative hypohalous solution has actually to be freshly prepared. It is particularly preferred to carry out the reaction as said oxidative solution is being prepared (see herein below).

According to a first variant, the buffered oxidative hypohalous solution is extemporaneously batchwise prepared outside the micro-reactor before its introduction into said micro-reactor. A buffering of a hypohalous solution to a desired pH value before (just before) its use can so be carried out. The prepared amount of the buffered oxidative hypohalous solution advantageously corresponds to the rapidly used amount of the buffered oxidative hypohalous solution. Said first variant is more particularly suitable for carrying out the process of the present disclosure for small scale applications, more particularly in a laboratory.

According to a second variant, which is preferred, the buffered oxidative hypohalous solution is extemporaneously prepared in-line. A hypohalous solution and a buffer solution can so be mixed just before the oxidation reaction in a mixing unit arranged upwardly (i.e., upstream) from the micro-reactor. It is quite possible for such a mixing unit to consist in a module of a micro-reactor. In such a case the micro-reactor used for carrying out the process of the present disclosure comprises a first module constituting a mixing unit (for the extemporaneous preparation of the buffered oxidative hypohalous solution) and at least one additional module for carrying out the oxidation reaction. The pH of the in-line buffered hypohalous solution can also be monitored on-line. Said second variant (according to which the oxidative solution is used as it being prepared) is highly advised when the reaction is carried out on an industrial scale (i.e. with large reaction volumes).

Here above have been indicated the three combined keys of the process of the present disclosure: carried out in a micro-reactor, with the buffered oxidative hypohalous solution extemporaneously prepared, at a buffered pH comprised between 7 and less than 8.5.

Concerning the required amount of catalyst, the following may be indicated in a non-limiting way. The conventional catalyst (nitroxide oxidation catalyst, see above) is generally used in less than 5 mol %, advantageously less than 2 mol %, very advantageously less than 0.5 mol %, in the process of the present disclosure (mol %=percentage of mol equivalent referred to the compound(s) present to oxidize).

It has to be noted that the process of the present disclosure for the oxidation of primary alcohol group(s), surprisingly requires less catalyst than the process of the prior art and that it can so be carried out with a concentration of catalyst less than 2 mol %, advantageously less than 0.5 mol %, very advantageously less than 0.07 mol %. Very good results have been obtained with the catalyst present at 0.05 mol %.

Considering now the co-catalyst (generally a source of Br⁻, such as KBr, Br₂, NaBr, R₄NBr (R being an alkyl group, for example), KBr being widely used) generally used in the prior art TEMPO-type oxidations of alcohols (see above as well as FIG. 1A), it is not totally excluded to use it in carrying out the process of the present disclosure. However the inventors have surprisingly noted that the oxidation process of the present disclosure does not require its presence. The addition of a co-catalyst can actually be quite useless in view of the good results obtained without it. So the process of the present disclosure is generally carried out without any co-catalyst, as shown in reaction diagram of FIG. 1B. This is particularly interesting, not only in reference to the realized savings but also in reference to the by-products able to be obtained when no co-catalyst is present (any halogen exchange with bromide (co-catalyst), when halogenated materials are involved, is avoided).

In the same way, it is not totally excluded to use a phase-transfer agent in carrying out the process of the present disclosure. However the presence of such a phase-transfer agent is in no way compulsory.

It has been here above specified that the oxidation process of the present disclosure is carried out in the presence of a buffered oxidative hypohalous solution, such an oxidative buffered hypohalous solution being known in the prior art. Such an oxidative buffered hypohalous solution advantageously consists in a bleach solution.

It has been here above specified that the oxidation process of the present disclosure is carried out in the presence of a nitroxide oxidation catalyst, such a catalyst being known in the prior art. So such a nitroxide oxidation catalyst is advantageously selected, for carrying out the process of the present disclosure, from the group consisting in the known nitroxide oxidation catalysts, from the group consisting in the (2,2,6,6-tetramethylpiperidin-1-yl)oxyl (TEMPO), in the TEMPO derivatives (such as HOT (4-hydroxy-TEMPO), 4-acetamido-TEMPO) and in the TEMPO or TEMPO derivatives on a support (such as PIPO (Polymer immobilized TEMPO)).

The used nitroxide oxidation catalyst is preferably TEMPO.

The process of the present disclosure is advantageously carried out with the buffered oxidative hypohalous solution as identified above and/or (very advantageously and) with the nitroxide oxidation catalyst as indentified above.

The process of the present disclosure is perfectly efficient at room temperature (between 15 and 25° C.). It is generally carried out at about 20° C. (between 18 and 22° C.).

The process according to the present disclosure can be carried out with or without solvent. It more particularly depends on the nature of the chemical compound(s) with the alcohol group(s) concerned.

The used solvent (if any) is advantageously selected from the group consisting in:
a. the alkanes such as pentane, hexane, heptane, octane, cyclohexane,
b. the aromatic solvents such as benzene, toluene, xylene,
c. the esters such as ethyl acetate, isopropyl acetate,
d. the chlorinated solvents such as dichloromethane, chloroform, carbon tetrachloride, ethylene tetrachloride,
e. the ketones such as acetone, butanone, pentanone, methyl isopropyl ketone, methyl isobutyl ketone, and
f. their mixtures.

The used solvent is very advantageously selected from the environmental friendly solvents.

Carrying out the conventional TEMPO-type oxidation in the above described conditions of reaction of the present disclosure allows obtaining the maximum yield very quickly. (Yield is a notion familiar to one skilled in the art. It is generally expressed in % as a ratio (in mass or in mole) of (1) the amount of the intended compound actually obtained to (2) the amount of the intended compound theoretically obtainable. The maximum yield is also a notion familiar to one skilled in the art. The reaction being complete, the yield reaches a maximum value and is not able to increase even if the experimental or the reaction conditions are longer maintained (and the yield is even able to decrease if the obtained compound is not stable)). By reaching maximum yield very rapidly, the process of the present disclosure allows the rate of the TEMPO-type oxidation to be surprisingly high. In a non-limitative way, it can here be indicated that the maximum yield value(s) is(are) generally of at least 50%, is(are) advantageously of at least 80% and that said maximum yield value(s) is(are) reached in a very short time (generally ≦3 min, indeed ≦1 min 30 s) and that at room temperature, without the use of any co-catalyst.

The chemical compound or at least one of the chemical compounds, the alcohol group of which or at least one of the alcohol groups of which is to be oxidized according to the process of the present disclosure, can be injected within the micro-reactor at a single injection point (generally at the entrance of the reactor) or at at least two injection points (a first one and at least one additional one), at least one of said at least two injection points being positioned downstream from a first injection point (generally positioned at the entrance of the reactor). Such an injection via at least two injection points is advantageously carried out to improve the control of the temperature of the reaction medium.

As indicated above, micro-reactors are known per se. However, one skilled in the art did not expect such a good result as that obtained according to the presently disclosed process used in the context of alcohol group(s) oxidation.

According to an advantageous variant, micro-reactors suitable for implementing the process of the present disclosure have at least two mixing zones along their reactant passage.

Such micro-reactors very advantageously have, successively along their length, at least two inlets, at least one for the buffered oxidative hypohalous solution, and at least one for the chemical compound(s) and TEMPO containing solution, an initial mixer passage portion, an initial dwell time passage portion having a volume of at least 0.1 ml and one or more additional mixer passage portions, each additional mixer passage portion followed immediately by a corresponding respective additional dwell time passage portion.

Within such a micro-reactor, the reactants are mixed in a first mixing zone, immediately after their injection. Then, they go through a dwell time passage portion. The sequence: mixing zone/dwell time zone is at least repeated twice. So, two mixings are at least carried out within such a micro-reactor. Such a micro-reactor is recommended for carrying out the process of the present disclosure.

Such micro-reactors have been described in EP1992404 by the applicant.

According to a second advantageous variant, micro-reactors suitable for implementing the process of the present disclosure are micro-reactors which ensure an essentially continuous mixing along their reactant passage.

Such micro-reactors very advantageously have a reactant passage which comprises multiple successive chambers, each chamber including a split of the reactant passage into at least two sub-passages, and a joining of the split passages, and a change of passage direction, of at least one of the sub-passages, of at least 90 degrees. According to a preferred variant, each of the multiple successive chambers, being immediately succeeded by another one of said chambers, further comprises a gradually narrowing exit which forms a corresponding narrowed entrance of the succeeding chamber and a splitting and re-directing wall oriented crossways to the immediately upstream passage and positioned immediately downstream of the chamber's entrance, the upstream side of said splitting and re-directing wall having a concave surface.

Within such micro-reactors, the mixing is essentially continuous.

Such micro-reactors have been described in EP2017000 by the applicant.

One skilled in the art knows generally how to optimize the compromise: efficiency of mixing/pressure drop in determining the structure of a micro-reactor. So, he will know generally how to optimize said compromise in determining the structure of the micro-reactors, used for carrying out the process of the present disclosure.

As explained above, the process of the present disclosure is able to be carried out in many contexts. It can be carried out to oxidize any (unprotected) alcohol group of the chemical compound(s) present in the reaction medium. It can be carried out to implement a selective oxidation. A selective oxidation is able to be managed by the setting of the residence time inside the reactor, supposing that there exists a sufficient difference of reactivity between some different alcohol groups (such a difference of reactivity being for example based on different steric hindrances and/or on a different chemical environment).

According to a preferred variant, the oxidation process of the present disclosure is carried out for oxidation of the alcohol group or all the alcohol groups of a single chemical compound. Within the scope of said preferred variant, it is advantageously carried out for oxidation of a single alcohol group of a single chemical compound; it is very advantageously carried out for oxidation of a single primary alcohol group of a single chemical compound (so as to generate an aldehyde; the oxidation of the 1-octanol and equivalents being an example).

According to another preferred variant, the oxidation process of the present disclosure is carried out for a selective oxidation of at least one alcohol group within a mixture comprising at least two chemical compounds, each having at least one alcohol group or of at least one alcohol group of a single chemical compound.

Within the scope of said preferred variant, it can be carried out for the selective oxidation of primary alcohol group(s) within a mixture of at least two chemical compounds, each having at least one alcohol group, or it can be carried out for the selective oxidation of primary alcohol group(s) of a chemical compound having said primary alcohol group(s) and at least one secondary alcohol group (the primary alcohol group(s) is(are) oxidized while the secondary alcohol group(s) is(are) not); it can be carried out for selective oxidation of at least one primary alcohol group of a chemical compound having primary alcohol groups (the selection is operated between the primary alcohol groups, in the presence or the absence of secondary alcohol group(s)) or for selective oxidation of at least one secondary alcohol group of a chemical compound having secondary alcohol groups (the selection is operated between the secondary alcohol groups, in the possible presence of aldehyde group(s) (resulting from the oxidation of at least a primary alcohol group of the chemical compound)).

No doubt that one skilled in the art has already understood the great interest of the process of the present disclosure. The main advantages of the process of the present disclosure are hereafter summarized:
a. the oxidation reaction goes rapidly to completion, generally in less than 3 minutes, sometimes within seconds,
b. the process is efficient at room temperature and without co-catalyst,
c. reduced amounts of catalyst can be used,
d. the process can be carried out for selective oxidations,
e. the process allows in-line monitoring of the pH to optimize the rate and yield of the reaction.

The claimed invention is now illustrated, in a non-limitative way, by the following examples and figures.

### BRIEF DESCRIPTION OF THE FIGURES

FIG. 1A (from R. Sheldon in Acc. Chem. Res. 2002, 35, 774) shows the proposed reaction mechanism for a TEMPO oxidation of a secondary alcohol with KBr as co-catalyst as generally understood.

FIG. 1B schematically shows the proposed reaction mechanism for the same TEMPO oxidation performed according to the preferred variant of the process of the present disclosure without any co-catalyst.

FIG. 2A schematically shows the process of the present disclosure (carried out in a micro-reactor without in-line pH adjustment).

FIG. 2B schematically shows the process of the present disclosure (carried out in another micro-reactor with in-line pH adjustment or control).

FIG. 3 shows the impact of the initial pH of the extemporaneously prepared buffered bleach solution on the yields and conversion, for a TEMPO oxidation carried out according to the present disclosure (see below example 1A) on the yield (Y) and conversion (C) percentage.

FIG. 4 shows the impact of the degradation of the (non-extemporaneously prepared) bleach solution on the yield (Y) and conversion (C) for a TEMPO oxidation as a function of time in minutes (see below example 1A′).

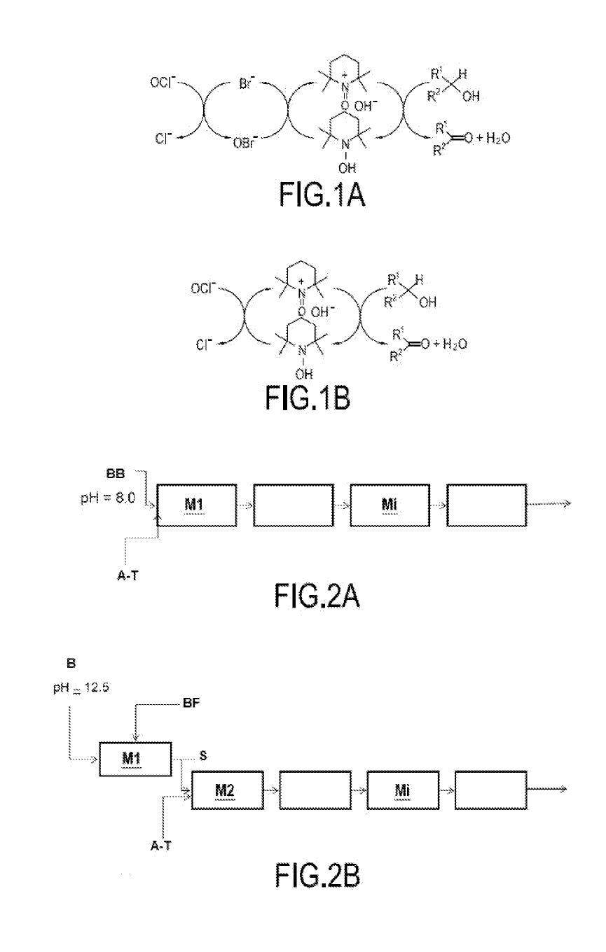

These figures are self-explanatory and/or have been commented in the above specification and/or are commented in the below examples.

### DETAILED DESCRIPTION OF SPECIFIC EXAMPLES

### Example A

The reaction is carried out in a round-bottomed flask with magnetic stirring (1000 rpm) at 0° C. (ice bath). The reaction takes place in a biphasic system. Alcohol (octanol-1:25 mmol, 3.94 ml, 3.25 g), TEMPO (1 mol %, 0.25 mmol, 39 mg) and an organic solvent (dichloromethane, 46 ml) are poured into the flask. Co-catalyst KBr (10 mol %, 2.5 mmol, 298 mg) is added next.

The solution of bleach is prepared at 5° C. The stock solution of bleach (10% w/w; 1.5 eq.; 22.6 ml) is diluted up to 50 ml with water. Then NaHCO₃ is added stepwise (three spatulas) to obtained a saturated solution. The pH is near 9. The bleach solution is added to the dichloromethane solution under vigorous stirring. The reaction is stirred with ice bath cooling. Samples are taken every minute during 5 minutes and then every 5 minutes. The samples are quenched with sodium thiosulfate and analyzed by GC(Gas Chromatography).

### Example B

The conditions are the same as in Example A but toluene is used instead of dichloromethane.

### Example BA

The conditions are the same as in Example B without co-catalyst KBr.

### Example BB

The conditions are the same as in Example BA. The reaction is performed at 20° C.

### Example BC

The conditions are the same as in Example BB, but 0.05 mol % TEMPO is used.

The results are given in following Table 1.

TABLE 1

| Example | Yield (%) | Time (min) |
|---|---|---|
| A | >98 | 4 |
| B | >98 | 5 |
| BA | 66 (reaction not complete) | 20 |
| BB | >98 | 15 |
| BC | 96 | 144 |

### Examples 1A, 1B, 1C

The micro-reactors used are glass micro-reactors with heart modules (said modules being schematically identified as Mi in FIGS. 2A and 2B), as described in (17).

The volume of the micro-reactor used in example 1A is about 3.15 ml (≈0.45 ml (internal volume of each module Mi)×7 (i=7))+the additional volume of tubing. Its reactant passage has a diameter of about 0.38 mm.

The volume of the micro-reactor used in example 1B is about 3.6 ml (≈0.45 ml (internal volume of each module Mi)×8 (i=8))+the additional volume of tubing; one module (the first one (M1) being used for in-line constituting the buffered bleach solution and seven other modules for the reaction (M2 to M8)). So the reaction volume is about 3.15 ml. The reactant passage has also a diameter of about 0.38 mm.

The volume of the micro-reactor used in example 1C is about 56.2 ml (≈7.03 ml (internal volume of each module M1)×8 (i=8))+the additional volume of tubing; one module (the first one (M1) being used for in-line constituting the buffered bleach solution and seven other modules for the reaction (M2 to M8)). So the reaction volume is about 49.2 ml. The reactant passage has a diameter between 0.95 and 1.25 mm. The modules of said micro-reactor are larger.

The feeding of the micro-reactors (more precisely, of the first module M1 of said micro-reactors) with the buffered bleaching solution is carried out according to two different ways:
a. for example 1A: without in-line pH adjustment, as shown on FIG. 2A and as explained in below example 1A;
b. for examples 1B and 1C: with in-line pH adjustment, as shown on FIG. 2B (said figure schematically shows both the “small” modules of the micro-reactor used in example 1B and the larger modules of the micro-reactor used in example 1C) and as explained in below examples 1B and 1C.

### Example 1A

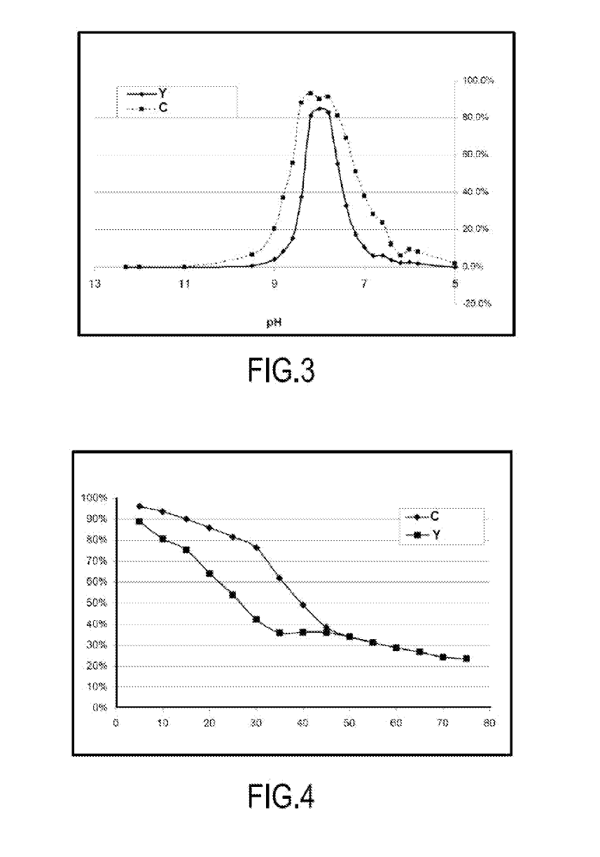

(FIGS. 2A and 3) (Including Comparative Data)

1-Octanol (c=0.5 mol/l) and 0.05% (molar) of TEMPO are dissolved in toluene, so constituting a first solution (the organic solution of Alcohol and Tempo labeled A-T on FIG. 2A).

For each trial, a small volume (about 50 ml) of a second solution—solution of buffered bleach at a given pH (see below Table 2) (feed labeled BB on FIG. 2A)—is extemporaneously prepared. (The presently preferred pH of 8.0, for certain embodiments, is shown in the figure, but not used in every experiment.)

The two solutions are pumped into the micro-reactor (into the first module M1 of the micro-reactor).

The total flow rate is always divided up in approximately two equal parts between organic and aqueous phases (between the first and the second solutions). The residence time is about 16 s.

The temperature is 20° C.

The results are given in the following Table 2 and shown in FIG. 3 (with the trace C representing conversion and the trace Y representing yield). They demonstrate the critical character of the pH.

TABLE 2

| | Yield | Conversion |
|---|---|---|
| pH | octanal (%) | 1-octanol (%) |
| 12.3 | 0.0 | 0.0 |
| 12 | 0.0 | 0.0 |
| 11 | 0.0 | 0.0 |
| 9.5 | 0.8 | 6.4 |
| 9 | 4.4 | 20.3 |
| 8.8 | 8.7 | 37.2 |
| 8.6 | 15.3 | 55.9 |
| 8.4 | 37.3 | 88.2 |
| 8.2 | 81.4 | 93.5 |
| 8 | 85.3 | 90.2 |
| 7.8 | 83.2 | 91.6 |
| 7.6 | 55.4 | 81.2 |
| 7.4 | 32.7 | 69.3 |
| 7.2 | 17.3 | 51.3 |
| 7 | 10.5 | 37.7 |
| 6.8 | 6.1 | 28.2 |
| 6.6 | 6.1 | 23.5 |
| 6.4 | 3.7 | 12.0 |
| 6.2 | 2.2 | 6.2 |
| 6 | 2.8 | 9.4 |
| 5.8 | 1.9 | 8.2 |
| 5 | 0.0 | 2.0 |
| 4.2 | 0.0 | 0.0 |
| 3.2 | 0.0 | 0.0 |

### Example 1B

### FIG. 2B

1-Octanol (c=1 mol/l) and 0.2% (molar) of TEMPO are dissolved in dichloromethane, thus constituting a solution for a first feed (i.e., feed 1 is this organic solution of Alcohol and TEMPO, labeled A-T on FIG. 2B)

A second feed (feed 2) is stock solution of bleach (c=11.2%) at a pH about 12.5, (labeled B in the figure). A third feed (feed 3) (labeled BF in the figure) is a 0.7 M phosphate buffer solution (pH 8), brought to pH 6.2 by slow and controlled addition of H₃PO₄ 85 wt. %. Feeds 2 and 3 (in a ratio buffer/bleach of 1.1) are mixed in one fluidic module (the first one M1 of the used micro-reactor) before passing a sensor S for monitoring pH. The mixture (the aqueous phase) is then injected into the second module M2 of the micro-reactor.

Feed 1 (A-T) is directly pumped to the module M2 where it is mixed with the aqueous phase.

The total ratio (aqueous/organic phase) is 1.33.

The temperature is 20° C.

The results obtained (conversion (%) and yield (%)) are indicated in the below Table 3 related to different experiments carried out at different residence time (Rt(s)).

TABLE 3

| Conversion* | Yield** | Rt |
|---|---|---|
| (%) | (%) | (s) |
| 100 | 87.8 | 73 |
| 98.7 | 90.6 | 57 |
| 92.2 | 84.1 | 49 |

*The conversion (%) is the ratio: difference between the starting octanol and the remaining octanol/the starting octanol.
**The yield (%) is the ratio: obtained aldhehyde/theoretically obtainable aldhehyde.

The results (conversion (%) and yield (%)) clearly show the interest of the present disclosure. The maximum yield is very rapidly obtained (in less than 80 s).

### Example 1C

### FIG. 2B

The micro-reactor used (of the type shown in FIG. 2B) has larger modules M1 than those used in examples 1A and 1B. It has been fed like the one of example 1B, as shown in FIG. 2B (two equal parts of the second and third feeds and two equal parts of the first and second solutions).

The two solutions are pumped into the micro-reactor. The total flow rate varies from 20 ml/min to 170 ml/min.

The temperature is 20° C.

The results obtained (conversion (%) and yield (%)) are indicated in the below Table 4 related to different experiments carried out at different flow rates (and consequently with different residence time (Rt(s)).

TABLE 4

| Conversion | Yield | Rt |
|---|---|---|
| (%) | (%) | (s) |
| 96% | 82% | 120.0 |
| 100% | 93% | 68.3 |
| 96% | 90% | 45.5 |
| 94% | 84% | 27.3 |
| 94% | 80% | 22.1 |
| 94% | 68% | 19.2 |
| 94% | 66% | 18.3 |
| 87% | 55% | 17.6 |

Said results clearly show the interest of the present disclosure. The maximum yield is very rapidly obtained (in less than 80 s).

### Example 1A′

### (Comparative Example) (FIGS. “2A” and 4)

The micro-reactor used is identical to the one of example 1A.

1-Octanol (c=1 mol/l) and 0.2% (molar) of TEMPO are dissolved in dichloromethane, so constituting a first solution (the organic solution A-T shown on FIG. 2A).

A large volume of a second solution—the one of buffered bleach (BB)—is not extemporaneously prepared (insofar as the trial lasts for 75 min) by mixing 1/1 volume of commercial bleach solution (10.6%, pH=12.5) and 0.75 M of NaH₂PO₄ solution. The initial pH of this stock solution of bleach (1.2 equivalent) is adjusted to 8.16 by slow and controlled addition of H₃PO₄ 85 wt. %.

The two solutions are pumped into the micro-reactor (into the first module M1 of the micro-reactor).

The residence time is 73 s and the ratio between aqueous and organic phases (between the first and the second solutions) is 1.3.

The temperature is 20° C.

The samples are taken out every five minutes. The results obtained for conversion (%) and yield (%) are indicated in the below Table 5. The pH of the buffered bleach solution feed stock is also indicated to highlight its rapid evolution correlated to bleach instability.

TABLE 5

| Time | Conversion* | Yield** | |
|---|---|---|---|
| (min) | (%) | (%) | pH |
| 0 | | | 8.16 |
| 5 | 96.2 | 88.9 | 8.06 |
| 10 | 93.7 | 80.7 | 7.96 |
| 15 | 90.1 | 75.5 | 7.87 |
| 20 | 86.0 | 64.3 | 7.79 |
| 25 | 81.6 | 54.0 | 7.69 |
| 30 | 76.5 | 42.4 | 7.6 |
| 35 | 62.1 | 36.0 | 7.49 |
| 40 | 49.3 | 36.2 | 7.43 |
| 45 | 38.7 | 36.1 | 7.36 |
| 50 | 34.0 | 34.0 | 7.28 |
| 55 | 31.4 | 31.4 | 7.22 |
| 60 | 28.9 | 28.9 | 7.17 |
| 65 | 26.8 | 26.8 | 7.11 |
| 70 | 24.4 | 24.4 | 7.07 |
| 75 | 23.6 | 23.6 | 7.04 |

*The conversion (%) is the ratio: difference between the starting 1-octanol and the remaining 1-octanol/the starting 1-octanol.
**The yield (%) is the ratio: obtained aldhehyde/theoretically obtainable aldhehyde.

The results (conversion (%) and yields (%)) are also shown in FIG. 4, as a function of time in minutes.

The curves of FIG. 4 clearly show high yield (Y) and conversion (C) only at the beginning of the experiment followed by a rapid degradation of the (non-extemporaneously) prepared bleach solution. Said degradation (monitored via the pH of the feed stock (see table 5)) reduced drastically the yield and conversion after few minutes of run.

### Example 2

### FIG. 2B

The micro-reactor used is identical to the one of example 1B.

Benzyl alcohol (c=1 mol/l) and 0.2% (molar) of TEMPO are dissolved in dichloromethane, thus constituting a solution for a first feed (feed 1=the organic solution (A-T) shown on FIG. 2B).

A second feed (feed 2) (B in the figure) is stock solution of bleach (c=11.2%) at a pH about 12.5. A third feed (feed 3) (BF in the figure) is a 0.7 M phosphate buffer (pH 8), which is brought to pH 6.2 by slow and controlled addition of H₃PO₄ 85% wt. Feeds 2 and 3 (in a ratio buffer/bleach of 1.1) are mixed in one fluidic module (the first one M1 of the used microreactor) before passing a control sensor S of set point pH (=8) and the mixture is then injected in the second module M2 of the micro-reactor. Feed 1 is directly pumped to the module M2 where it is mixed with aqueous phase.

The total ratio (aqueous/organic phase) is 1.33.

The temperature is 20° C.

The results obtained (conversion (%) and yield (%)) are indicated in the Table 6 below related to different experiments carried out at different residence time (Rt(s)).

TABLE 6

| Rt | Conversion* | Yield** |
|---|---|---|
| (s) | (%) | (%) |
| 73 | 100 | 100 |
| 57 | 100 | 100 |
| 49 | 100 | 100 |

*The conversion (%) is the ratio: difference between the starting benzyl alcohol and the remaining benzyl alcohol/the starting benzyl alcohol.
**The yield (%) is the ratio: obtained aldhehyde/theoretically obtainable aldhehyde.

### Example 3

### FIG. 2B

The micro-reactor used is identical to the one of example 1B.

Cyclohexanol (c=1 mol/l) and 4% (molar) of TEMPO are dissolved in dichloromethane, thus constituting a solution for a first feed (feed 1=the organic solution (A-T) shown on FIG. 2B).

A second feed (feed 2) is stock solution of bleach (c=10%) at a pH about 12.5 (labeled B on FIG. 2B). A third feed (feed 3) (labeled BF on FIG. 2B) is a 0.85 M phosphate buffer solution (pH 8) which is brought to pH 6.5 by slow and controlled addition of H3PO4 85% wt. Feeds 2 and 3 (in a ratio buffer/bleach of 0.8) are mixed in one fluidic module (the first one M1 of the used micro-reactor) before passing a control sensor S with target of pH (=8) and the mixture is then injected in the second module M2 of the micro-reactor. Feed 1 is directly pumped to the module M2 where it is mixed with aqueous phase.

The total ratio (aqueous/organic phase) is 1.33.

The temperature is 20° C.

The results obtained (conversion (%) and yield (%)) are indicated in the below Table 7 related to different experiments carried out at different residence time (Rt(s)).

TABLE 7

| Rt | Conversion* | Yield** |
|---|---|---|
| (s) | (%) | (%) |
| 73 | 96.1 | 93.3 |
| 57 | 94.3 | 94.3 |
| 49 | 92.8 | 92.8 |
| 38 | 91.5 | 91.5 |

*The conversion (%) is the ratio: difference between the starting cyclohexanol and the remaining cyclohexanol/the starting cyclohexanol.
**The yield (%) is the ratio: obtained ketone/theoretically obtainable ketone.

### Example 4

### FIG. 2B

The micro-reactor used is identical to the one of example 1B.

1-Octanol (c=0.5 mol/l), 2-Octanol (c=0.5 mol/l) and 0.2% (molar) of TEMPO are dissolved in dichloromethane, thus constituting a solution for the first feed (feed 1=the organic solution (labeled A-T in the figure)).

A second feed (feed 2) (labeled B) is stock solution of bleach (c=10.6%) at a pH about 12.5. A third feed (feed 3) (labeled BF) is a 0.75 M phosphate buffer solution (pH 8), which is brought to pH 6.3 by slow and controlled addition of H₃PO₄ 85 wt. %. Feeds 2 and 3 (in a ratio buffer/bleach of 1) are mixed in one fluidic module (the first one M1 of the used micro-reactor) before passing a pH control sensor S (with target pH of 8) and the mixture is then injected in the second module M2 of the micro-reactor. Feed 1 is directly pumped to the module M2 where it is mixed with the aqueous phase.

The total ratio (aqueous/organic phase) is 1.33.

The temperature is 20° C.

The results obtained (conversion (%) and yield (%)) are indicated in the below Table 8 related to an experiment carried out at 49 s of residence time (Rt(s)).

TABLE 8

| Conversion* | Yield** | Conversion* | Yield** | |
|---|---|---|---|---|
| 1-octanol | 1-octanol | 2-octanol | 2-octanol | |
| (%) | (%) | (%) | (%) | Rt (s) |
| 98.5 | 86.4 | 28 | 28 | 49 |

*The conversion (%) is the ratio: difference between the starting alcohol and the remaining alcohol/the starting alcohol.
**The yield (%) is the ratio: obtained aldhehyde/theoretically obtainable aldhehyde.

This non-optimized experiment shows that it will be possible to carry out a selective oxidation with a shorter residence time.

1. A process for oxidation of at least one alcohol group of at least one chemical compound to the corresponding carbonyl group, in the presence of a buffered oxidative hypohalous solution and of a nitroxide oxidation catalyst, wherein it is carried out within a micro-reactor and wherein the buffered oxidative hypohalous solution is extemporaneously prepared at a buffered pH comprised between 7 and less than 8.5 (7≦pH<8.5), advantageously comprised between 7.5 and 8.4 (7.5≦pH≦8.4).

2. The process according to claim 1 wherein it is carried out for the oxidation of primary alcohol group(s) and wherein the buffered oxidative hypohalous solution is extemporaneously prepared at a buffered pH between 7.8 and 8.2 (7.8≦pH≦8.2).

3. The process according to claim 1 wherein it is carried out for the oxidation of secondary alcohol group(s) and wherein the buffered oxidative hypohalous solution is extemporaneously prepared at a buffered pH between 8 and 8.4 (8≦pH≦8.4).

4. The process according to claim 1 wherein the buffered oxidative hypohalous solution is extemporaneously batchwise prepared outside the micro-reactor before its introduction into said micro-reactor.

5. The process according to claim 1 wherein the buffered oxidative hypohalous solution is extemporaneously in-line prepared.

6. The process according to claim 1 wherein it is carried out with a concentration of the catalyst less than 5 mol %, advantageously less than 2 mol %, very advantageously less than 0.5 mol %.

7. The process according to claim 1 wherein it is carried out for the oxidation of primary alcohol group(s) with a concentration of the catalyst less than 2 mol %, advantageously less than 0.5 mol %, very advantageously less than 0.07 mol %.

8. The process according to claim 1, wherein it is carried out without any co-catalyst.

9. The process according to claim 1 wherein the oxidative buffered hypohalous solution is a bleach solution and/or the nitroxide oxidation catalyst is selected from the group consisting in the (2,2,6,6-tetramethylpiperidin-1-yl)oxyl (TEMPO), in the TEMPO derivatives, and in the TEMPO or TEMPO derivatives on a support.

10. The process according to claim 1 wherein the chemical compound or at least one of the chemical compounds is injected within the micro-reactor at at least two injection points, at least one downstream from a first injection point.

11. The process according to claim 1 wherein it is carried within a micro-reactor with at least two mixing zones along the reactant passage.

12. The process according to claim 11 wherein said reactant passage has, successively along its length, at least two inlets, at least one for the buffered oxidative hypohalous solution, and at least one for the chemical compound(s) and TEMPO containing solution, an initial mixer passage portion, an initial dwell time passage portion having a volume of at least 0.1 ml and one or more additional mixer passage portions, each additional mixer passage portion followed immediately by a corresponding respective additional dwell time passage portion.

13. The process according to claim 1 wherein it is carried within a micro-reactor with essentially continuous mixing along the reactant passage.

14. The process according to claim 13 wherein said reactant passage comprises multiple successive chambers, each chamber including a split of the reactant passage into at least two sub-passages, and a joining of the split passages, and a change of passage direction, of at least one of the sub-passages, of at least 90 degrees.

15. The process according to claim 1 wherein it is carried out for oxidation of the alcohol group or all the alcohol groups of a single chemical compound, wherein it is advantageously carried out for oxidation of a single alcohol group of a single chemical compound, wherein it is very advantageously carried out for oxidation of a single primary alcohol group of a single chemical compound.

16. The process according to claim 1 wherein it is carried out for a selective oxidation of at least one alcohol group within a mixture comprising of at least two chemical compounds, each having at least one alcohol group or of at least one alcohol group of a single chemical compound.

17. The process according to claim 16 wherein it is carried out for the selective oxidation of primary alcohol group(s) within a mixture comprising of at least two chemical compounds, each one having at least one alcohol group, or for the selective oxidation of primary alcohol group(s) of a chemical compound having said primary alcohol group(s) and at least one secondary alcohol group, or for selective oxidation of at least one primary alcohol group of a chemical compound having primary alcohol groups or for selective oxidation of at least one secondary alcohol group of a chemical compound having secondary alcohol groups.
